(19) United States
(12) Patent Application Publication
(10) Pub. No.: US 2009/0131848 A1
(43) Pub. Date: May 21, 2009

(54) METHOD FOR IMPLANTING A TRANSCORNEAL IMPLANT THROUGH A PARACENTESIS T-INCISION, AND TRANSCORNEAL IMPLANT SO IMPLANTED

(75) Inventor: Margaret Taylor, Groton, MA (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: Becton, Dickinson and Company, Franklin Lakes, NJ (US)

(21) Appl. No.: 11/941,150

(22) Filed: Nov. 16, 2007

(51) Int. Cl. A61M 1/00; A61F 9/01

(52) U.S. Cl. 604/8; 606/166

(57) ABSTRACT

A method for implanting a transcorneal implant having a cap, a foot, and a body connecting the cap and the foot, the body having a lesser circumference than either the cap or the foot, the method including the operations: creating a first corneal incision to create a tunnel, such that the a far end of the first incision enters the anterior chamber; creating a second corneal incision opening a roof of the tunnel to an end point past the far end of the first incision and creating flaps; guiding the implant to the end point, so that the foot of the implant is under the flaps; rotating the implant until the foot is entirely within the anterior chamber; and rotating the implant back to a neutral position, such that a portion of the foot is beneath a floor of the tunnel.

### BACKGROUND OF THE INVENTION

1. Field of the Invention

The present invention relates to a method for implanting an ophthalmic implant, and more particularly, to a method for implanting a transcorneal implant through a paracentesis T-incision.

2. Description of the Related Art

Glaucoma, a condition caused by optic nerve cell degeneration, is the second leading cause of preventable blindness in the world today. In the human eye, aqueous humor is a transparent liquid that is constantly secreted by the ciliary body around the lens and flows into the region of the eye between the cornea and the lens, the anterior chamber. The trabecular meshwork provides the means by which the aqueous humor naturally drains from the anterior chamber. A major symptom of glaucoma is a high intraocular pressure, or “IOP,” which is caused by the trabecular meshwork failing to drain enough aqueous humor fluid from within the eye.

Conventional glaucoma therapy has been directed at protecting the optic nerve and preserving visual function by attempting to lower IOP using various methods, such as using drugs or surgery methods, including trabeculectomy and the use of implants. Trabeculectomy is a very invasive surgical procedure in which no device or implant is used. Typically, a surgical procedure is performed to puncture or reshape the trabecular meshwork by surgically creating a channel, thereby opening the sinus venosus.

Another surgical technique typically used involves the use of implants, such as stents or shunts, which are positioned within the eye and are typically relatively large. Such devices are implanted during any number of surgically invasive procedures, and serve to relieve internal eye pressure by permitting aqueous humor fluid to flow from the anterior chamber, through the sclera, and into a conjunctive bleb over the sclera. These procedures are very labor intensive for the surgeons and may be subject to failure due to scarring and cyst formations.

Another problem often related to the treatment of glaucoma with drugs relates to the challenge of delivering drugs to the eye. Current methods of delivering drugs to the eye are not as efficient or effective as desirable. Most drugs for the eye are applied in the form of eye drops, which have to penetrate through the cornea and into the eye. Drops are an inefficient way of delivering drugs; much of the drug never reaches the inside of the eye. Another treatment procedure includes injections. Drugs may be injected into the eye, but this is often traumatic and the eye typically needs to be injected on a regular basis.

One solution to the problems encountered with treatment of glaucoma using drops and injections involves the use of a transcorneal shunt, as disclosed herein. The transcorneal shunt is designed to be an effective means to reduce the intraocular pressure in the eye by shunting aqueous humor fluid from the anterior chamber of the eye. Surgical implantation of a transcorneal shunt is less invasive and quicker than other surgical options because the device is intended for implantation in the clear cornea. The transcorneal shunt drains aqueous humor fluid through the cornea to the tear film, rather than to the trabecular meshwork.

Additional details of ophthalmic shunts can be found, for example, in U.S. patent application Ser. No. 10/857,452, entitled “Ocular Implant and Methods for Making and Using Same,” filed Jun. 1, 2004 and published Jun. 2, 2005 under U.S. Publication No. 2005/0119737 A1, as well as International Patent Application No. PCT/US01/00350, entitled “Systems And Methods For Reducing Intraocular Pressure”, filed on Jan. 5, 2001 and published on Jul. 19, 2001 under the International Publication No. WO 01/50943. Details of ophthalmic shunts can also be found in U.S. Pat. No. 5,807,302, entitled “Treatment of Glaucoma,” filed Apr. 1, 1996 and issued Sep. 15, 1998. The entire contents of these applications and patent are incorporated herein by reference.

### SUMMARY OF THE INVENTION

Accordingly, it is an aspect of the present invention to provide the ability to implant a shunt through a cornea while minimizing risk of damage to the iris or lens, and providing a substantially leak-free seal around the shunt.

The foregoing and/or other aspects of the present invention are achieved by providing a method for implanting a transcorneal shunt having a cap, a foot, and a body connecting the cap and the foot, the body having a lesser circumference than either the cap or the foot. The method may include: creating a first corneal incision to create a tunnel, such that the a far end of the first incision enters the anterior chamber; and creating a second corneal incision opening a roof of the tunnel to an end point past the far end of the first incision and creating flaps. The method may also include: guiding a shunt to the end point, so that the foot of the shunt is under the flaps; rotating the shunt until the foot is entirely within the anterior chamber; and rotating the shunt back to a neutral position, such that a portion of the foot is beneath a floor of the tunnel.

The foregoing and/or other aspects of the present invention are also achieved by providing a method for creating a corneal paracentesis for implantation of a transcorneal shunt. The method may include: creating a first corneal incision to create a tunnel, such that the a far end of the first incision enters the anterior chamber; and creating a second corneal incision opening a roof of the tunnel to an end point past the far end of the first incision, to define an implantation location for the shunt and complete the paracentesis.

The foregoing and/or other aspects of the present invention are also achieved by providing a corneal paracentesis T-incision, including: a clear corneal incision creating a tunnel from a surface of the clear cornea to the anterior chamber; and a second incision, creating flaps and opening a roof of the clear corneal incision tunnel to an end point past a far end of the clear corneal incision tunnel.

The foregoing and/or other aspects of the present invention are also achieved by providing a corneal implant implanted in a paracentesis T-incision, the paracentesis T-incision including a clear corneal incision creating a tunnel from a surface of the clear cornea to the anterior chamber, and a second incision, creating flaps and opening a roof of the clear corneal incision tunnel to an end point past a far end of the clear corneal incision tunnel. The implant has a body connecting a cap and a foot, the foot is disposed within the anterior chamber, a portion of the foot is disposed beneath a floor of the clear corneal incision tunnel, and the flaps are tucked under the cap.

Additional and/or other aspects and advantages of the present invention will be set forth in part in the description that follows and, in part, will be apparent from the description, or may be learned by practice of the invention.

### BRIEF DESCRIPTION OF THE DRAWINGS

The above and/or other aspects and advantages of embodiments of the invention will become apparent and more readily appreciated from the following detailed description, taken in conjunction with the accompanying drawings, of which:

FIG. 1 illustrates a cross section of an ophthalmic shunt;

FIG. 2 is a photograph of an ophthalmic shunt

FIG. 3 illustrates a cross section of the ophthalmic shunt of FIG. 1 implanted in a cornea;

FIG. 4 illustrates a gape around an implanted shunt;

FIG. 5 is a representation of a cross section of corneal tissue;

FIGS. 6A and 6B illustrate a paracentesis T-incision according to an embodiment of the present invention;

FIGS. 7A and 7B illustrate creation of a clear corneal tunnel incision according to an embodiment of the present invention;

FIG. 8A illustrates opening a roof of the tunnel of FIG. 7B using scissors;

FIG. 8B illustrates a resulting paracentesis T-incision according to an embodiment of the present invention;

FIGS. 9A-9D illustrate opening the roof of the tunnel of FIG. 7 using a blade; and

FIGS. 10A-10G illustrate implanting a transcorneal shunt according to an embodiment of the present invention.

### DETAILED DESCRIPTION OF EMBODIMENTS OF THE PRESENT INVENTION

Reference will now be made in detail to embodiments of the present invention, examples of which are illustrated in the accompanying drawings, wherein like reference numerals refer to the like elements throughout. The embodiments described explain the present invention by referring to the figures.

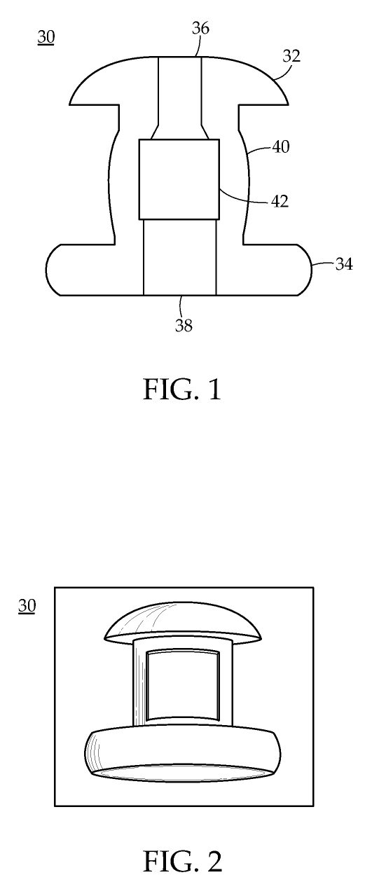

FIG. 1 illustrates a cross section of an ophthalmic implant 30 in accordance with an embodiment of the present invention, for example, a transcorneal shunt 30. The shunt 30 has a head or cap 32 and a foot 34, each having a hole 36, 38 therein. A body 40 forms a conduit that extends between the cap 32 and foot 34. The shunt 30 also includes a filter 42 to regulate the flow of aqueous humor from the anterior chamber and limit the ingress of harmful microorganisms into the anterior chamber. As can be seen, the body 40 has a smaller circumference than either the cap 32 or the foot 34. For example, the cap 32 may have a diameter of approximately 0.055 in. and a height of approximately 0.012 in., the foot 34 may have a diameter of approximately 0.065 in. and a height of approximately 0.013 in., and the body 40 may have a diameter of approximately 0.034 in. Additionally, a distance between the cap 32 and the foot 34, or a length of the body 40, may be approximately 0.030 in. or 0.036 in. to account for varying corneal thicknesses.

If the shunt 30 is made of, for example, hydrogel, these sizes represent final or hydrated dimensions of the shunt 30, since in a dehydrated state, the dimensions would be reduced by about 22%. Other materials that can be used to manufacture ophthalmic shunt 30 include: elastomeric materials, such as silicone rubber and polyurethane; glass; ceramic; polycarbonate; acrylic resin; stainless steel; titanium; silver; gold; and platinum. Materials that can be used to manufacture the filter 42 include: silicon, polymers, or sintered metals, such as titanium.

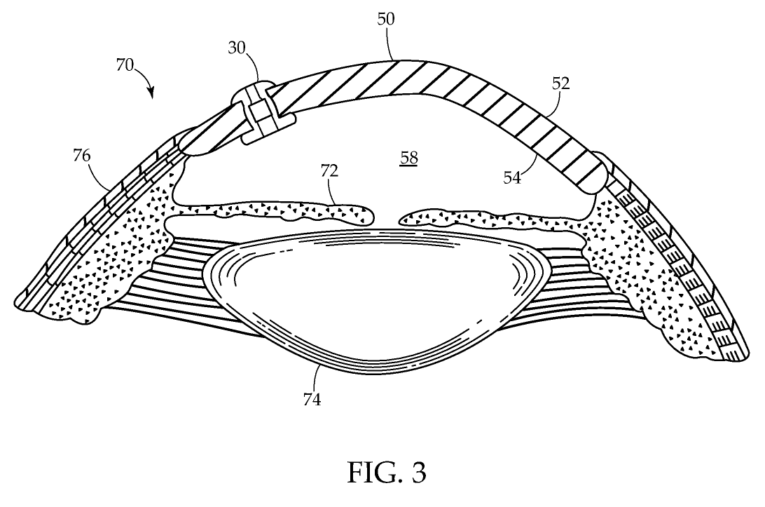

FIG. 2 is a photograph of an ophthalmic shunt 30. FIG. 3 illustrates a cross section of the ophthalmic shunt 30 of FIG. 1 implanted in a cornea 50. One method for inserting or implanting the transcorneal shunt 30 in the cornea 50 is through a small, straight incision, approximately perpendicular to the cornea 50. Using this method, ideally, a surgeon (or other suitably trained person—for brevity, such a person will hereinafter be referred to as a surgeon or a user) selectively sizes the incision to allow the foot 34 to be manipulated through the incision, and yet prevent both the head 32 and the foot 34 from passing through once the shunt 30 is in place, thereby securing the shunt 30 in position. Once the foot 34 is placed in the incision, the surgeon pushes the shunt 30 down. Then, ideally, head 32 anchors the shunt 30 on an outside surface 52 of the cornea 50, and the foot 34 anchors the shunt 30 on an inside surface 54 of the cornea 50.

But since the incision is substantially a straight line, and is larger than a diameter of the foot 34, as shown, e.g., in FIG. 4, after insertion using such a method, there can be a gape 56 around the body 40 of the shunt 30. Leakage of aqueous humor from the anterior chamber 58 (see e.g., FIG. 2) may result from such a gape 56.

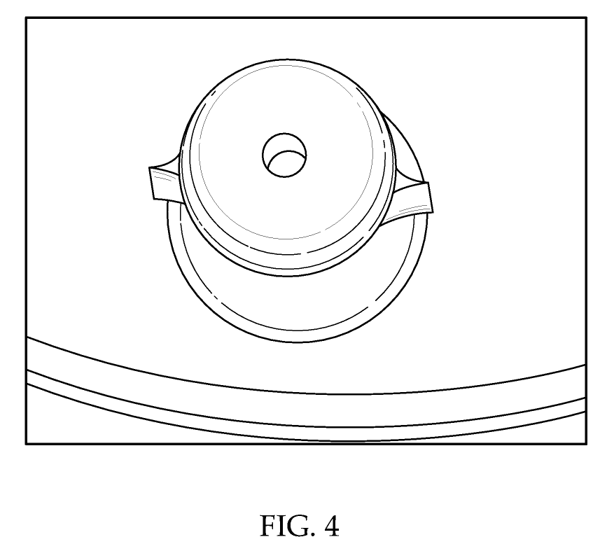

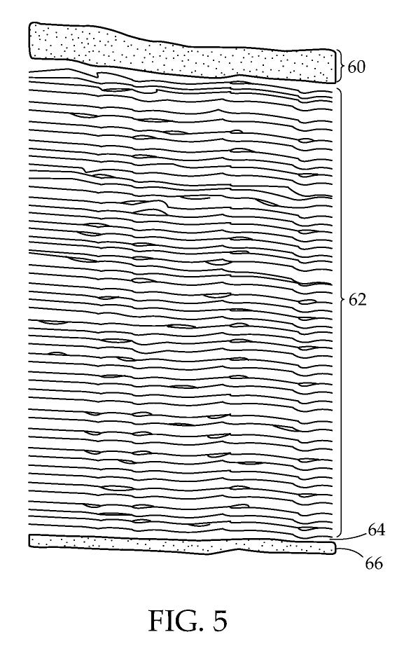

Additionally, implantation using this incision technique may not always result in optimal shunt placement. FIG. 5 is a representation of a cross section of corneal tissue. As shown in FIG. 5, the human cornea has four layers: corneal epithelium 60, a thin multicellular layer of fast-growing and easily-regenerated cells; corneal stroma 62 (or stroma), a thick, transparent middle layer; Descemet's membrane 64 (or posterior limiting membrane), a thin layer that serves as a basement membrane of the corneal endothelium; and the corneal endothelium 66, a monolayer of specialized cells that lines the posterior surface of the cornea and faces the anterior chamber 58. Descemet's membrane is more resistant than the stroma, so when employing the above-described method, the foot 34 may sometimes get stuck in the stroma, and not pass fully through the cornea.

Further, using the above-described method, during the creation of a perpendicular corneal incision close to the limbus 70, there is a risk that the blade may hit the iris 72 or lens 74 (see FIG. 3), causing complication from the procedure, such as cataract formation.

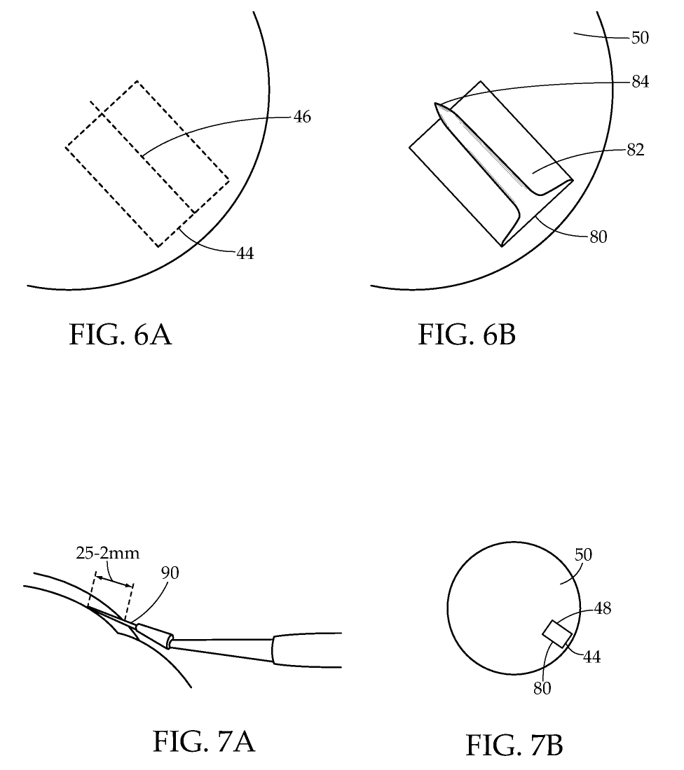

Accordingly, a new method has been developed for implanting a transcorneal shunt via a paracentesis T-incision. FIGS. 6A and 6B illustrate a paracentesis T-incision according to an embodiment of the present invention. A three-dimensional architecture of a paracentesis T-incision is created with two incisions: first, as shown in FIG. 6A, a corneal tunnel incision 44 enabling the foot 34 to be implanted into the anterior chamber; and a second incision 46, which can be extended slightly beyond the entry wound of the tunnel incision 44, unroofing the tunnel, enabling the shunt 30 to be placed in the correct position, and also providing a seal around the body 40 where the first incision 44 gaps. As shown in FIG. 6B, a completed paracentesis T-incision includes an elongated tunnel 80, flaps 82, and a gap 84 defining an implantation location for a transcorneal shunt, such as shunt 30. According to one embodiment, the tunnel 80 is created in the clear cornea.

Dr. Howard Fine introduced a clear corneal incision for use in penetrating keratoplasty, or corneal transplantation. Clear corneal incisions have also been employed in cataract surgeries, e.g., phacoemulsification, in which the cataract is emulsified by a vibrating needle, and then aspirated. One of the primary advantages of a clear corneal incision is that sutures are generally not necessary to close the wound.

FIGS. 7A and 7B illustrate creation of a paracentesis incision 44, more specifically, a clear corneal tunnel incision 44 according to an embodiment of the present invention. As shown in FIG. 7A, according to one embodiment, the surgeon angles a blade 90 used to create this first incision 44 toward an anterior presentation with respect to the eye. Such a presentation and incision 44 provides a tunnel 80 that is approximately parallel to the iris, thereby substantially reducing the risk that the blade will hit the iris or the lens. As shown in FIG. 7B, this first incision 44 begins close to the limbus. According to one embodiment, the surgeon creates the first incision 44 using a cataract style blade to make a clear corneal incision 44 approximately 1.5 mm-2.0 mm in length, such that the wound enters the anterior chamber approximately 2.0 mm from the superior limbus (between 11 and 1 o'clock).

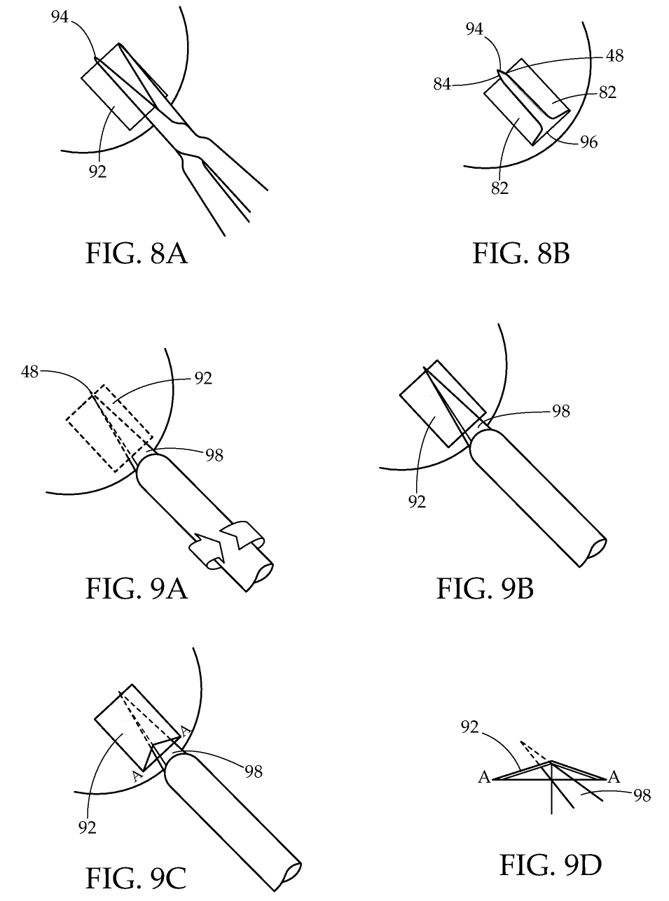

FIG. 8A illustrates opening a roof of the tunnel 80 using scissors, and FIG. 8B illustrates a resulting paracentesis T-incision according to an embodiment of the present invention. And FIGS. 9A-9D illustrate opening the roof of the tunnel 80 using a blade. In FIG. 8A, the roof 92 of tunnel 80 is approximately bisected using scissors, such as Vannas scissors, or the like, creating flaps 82. As shown in FIG. 8, the surgeon bisects the roof of the tunnel 80 to an end point 94 past a far end 48 of the first incision 44 (i.e., the wound edge through the corneal endothelium), creating gap 84. According to one embodiment, the second incision 46 extends approximately 0.5 mm past the far end 48 of the first incision 44. It is advisable that the surgeon be careful not to damage a floor 96 of the tunnel 80 when creating the second incision 46.

In FIGS. 9A-9D, instead of scissors, a blade 98 is employed to create the second incision 46. Initially, as shown in FIG. 9A, the surgeon inserts the blade 98 (e.g., a 15° blade, or the like) substantially parallel to the roof 92 of the tunnel 80, being careful not to widen the tunnel 80 or cut tissue. According to one embodiment, the surgeon inserts a tip of the blade 98 approximately 0.5 mm past the far end 48 of the first incision 44 (i.e., the wound edge through the corneal endothelium). Then, the surgeon rotates the blade 98 about a longitudinal axis thereof (FIG. 9B). After rotating the blade 98 approximately 90 so that a cutting edge of the blade 98 faces the roof 92 of the tunnel 80 approximately perpendicularly (FIGS. 9C and 9D), the surgeon applies light upward pressure to create the second incision 46. The resulting paracentesis T-incision from using scissors or from using a blade should be substantially the same.

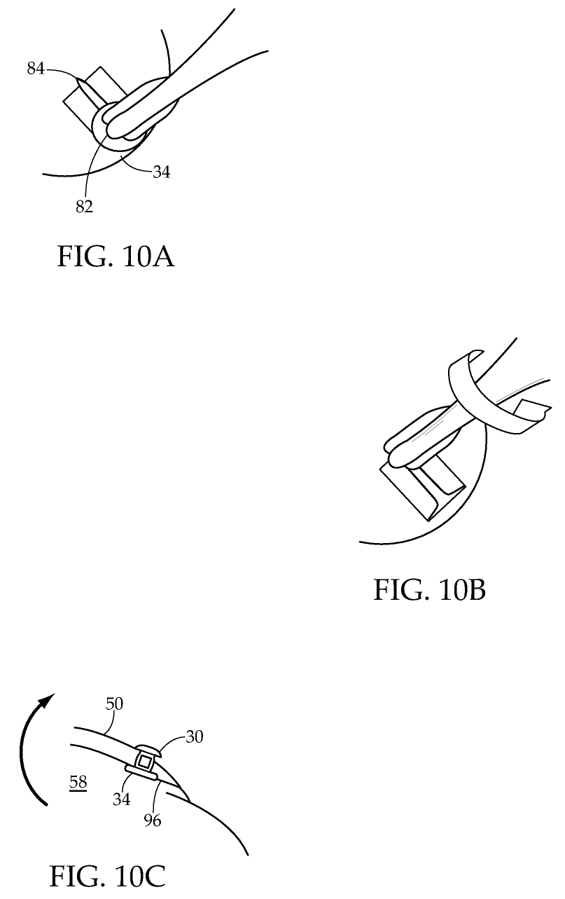

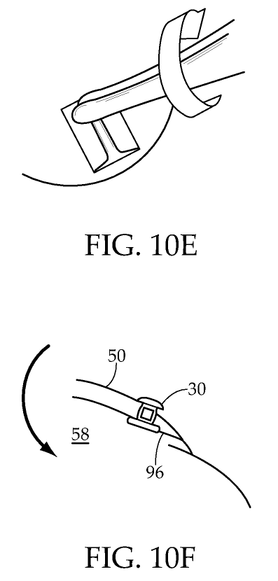

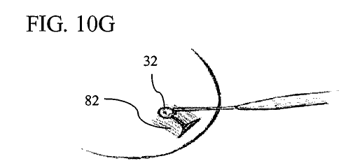

FIGS. 10A-10G illustrate implanting a transcorneal shunt according to an embodiment of the present invention. As shown in FIG. 10A, the surgeon guides the shunt 30 into the paracentesis T-incision until the body 40 of the shunt 30 reaches the gap 84 (i.e. to the end point 94 of the second incision—see FIG. 10B), such that the foot 34 of the shunt 30 is under the flaps 82. By “under,” this application means radially closer to a center of the eye, with respect to a radius of an approximated sphere representing the eye. According to one embodiment, as shown in FIG. 10C, at this point, the body 40 of the shunt 30 is positioned against the end point 94 of the second incision, and a portion of the foot 34 is positioned in the anterior chamber 58, and a portion of the foot 34 is positioned adjacent the floor 96 of the tunnel 80.

Next, the surgeon rotates the shunt 30 up to about 90°, rotating the cap 32 towards the limbus. According to one embodiment, an axis for this rotation is substantially perpendicular to the second incision. The direction of rotation is represented by the rotational arrows in FIGS. 10B and 10C. During this first rotation, the surgeon may feel a “pop” as an edge of the foot 34 farthest away from the end point 94 passes under the corneal layers (i.e. the floor 96 of the tunnel 80) and into the anterior chamber. At this point, the foot 34 should be entirely within the anterior chamber 58.

Then, the surgeon rotates the shunt 30 back by up to about 90° to a neutral position (illustrated by the rotational arrows of FIGS. 10D and 10E), such that a portion of the foot 34 is positioned under the floor 96 of the tunnel 80 (as shown, e.g., in the side view illustrated in FIG. 10E) and the entire foot 34 is within the anterior chamber 58.

Following this second rotation, as shown in FIG. 10F, the surgeon can check the seating of the shunt 30 by attempting to axially rotate, or pivot the shunt 30 (the axis of rotation being approximately co-linear with the passage through the shunt 30). A properly seated shunt 30 should pivot freely. If the shunt 30 does not pivot freely, then the foot 34 is likely caught in the stroma 62. If so stuck, the surgeon can re-attempt the first and second rotations, or simply apply downward pressure. Such manipulation can be accomplished with e.g., a Sinskey hook, or the like.

Once the shunt 30 is seated, as shown in FIG. 10G, the surgeon tucks the flaps 82 under the cap 32 of the shunt 30, to create a seal around the body 40 of the shunt 30.

After the shunt 30 has been implanted, a suture may be used for placement of the corneal tissue. A substantially leak-free seal around the device, however, will likely be achieved without the use of the suture.

Accordingly, after the tucking of the flaps 82, in accordance with an embodiment of the present invention, a corneal implant (e.g. shunt 30) is implanted in a paracentesis T-incision. The paracentesis T-incision includes a clear corneal incision creating a tunnel from a surface of the clear cornea to the anterior chamber, and a second incision, creating flaps and opening a roof of the clear corneal incision tunnel to an end point past a far end of the clear corneal incision tunnel. The implant has a body connecting a cap and a foot, and the foot is disposed within the anterior chamber. Additionally, a portion of the foot is disposed beneath a floor of the clear corneal incision tunnel, and the flaps are tucked under the cap.

As shown and described herein, the new paracentesis T incision enables a foot of a corneal shunt to be implanted through the cornea without the seating issues and injuries to the iris and/or lens that can occur using previous methods, and the flaps created by unroofing the clear corneal incision cover the wound around the device to create a substantially leak-free seal.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. A method for implanting a transcorneal shunt having a cap, a foot, and a body connecting the cap and the foot, the body having a lesser circumference than either the cap or the foot, the method comprising:
creating a first corneal incision to create a tunnel, such that the a far end of the first incision enters the anterior chamber;
creating a second corneal incision opening a roof of the tunnel to an end point past the far end of the first incision and creating flaps;
guiding the implant to the end point, so that the foot of the implant is under the flaps;
manipulating the implant until the foot is entirely within the anterior chamber.

2. The method according to claim 1, wherein the manipulating the shunt until the foot is entirely within the anterior chamber comprises rotating the implant until the foot is entirely within the anterior chamber.

3. The method according to claim 2, further comprising rotating the implant back to a neutral position, such that a portion of the foot is beneath a floor of the tunnel.

4. The method according to claim 1, further comprising tucking the flaps under the cap.

5. The method according to claim 1, further comprising freely pivoting the implant in the neutral position, to confirm proper insertion of implant.

6. The method according to claim 1, wherein the rotating of the implant occurs substantially perpendicular to the second corneal incision.

7. The method according to claim 1, wherein the first incision is a clear corneal incision.

8. The method according to claim 7, wherein the clear corneal incision is substantially parallel to the iris

9. The method according to claim 7, wherein the clear corneal incision is approximately 1.5 mm to 2.0 mm long.

10. The method according to claim 7, wherein the far end of the clear corneal incision is approximately 2.0 mm from the superior limbus.

11. The method according to claim 1, wherein the end point is approximately 0.5 mm past the far end of the first incision.

12. The method according to claim 1, wherein the creating the second corneal incision comprises using scissors to open the roof of the tunnel.

13. The method according to claim 1, wherein the creating the second corneal incision comprises:
inserting a blade into the tunnel such that the blade is substantially parallel to the roof of the tunnel;
rotating the blade approximately 90 degrees such that a cutting edge of the blade faces the roof of the tunnel; and
using the cutting edge of the blade to open the roof of the tunnel.

14. The method according to claim 1, wherein during the creating of the first incision, a blade of an instrument used to create the first incision is angled toward an anterior presentation with respect to the eye.

15. A method for creating a corneal paracentesis for implantation of a transcorneal implant, the method comprising:
creating a first corneal incision to create a tunnel, such that the a far end of the first incision enters the anterior chamber;
creating a second corneal incision opening a roof of the tunnel to an end point past the far end of the first incision, to define an implantation location for the implant and complete the paracentesis.

16. The method according to claim 15, wherein the first incision is a clear corneal incision.

17. The method according to claim 16, wherein the clear corneal incision is substantially parallel to the iris

18. The method according to claim 16, wherein the clear corneal incision is approximately 1.5 mm to 2.0 mm long.

19. The method according to claim 16, wherein the far end of the clear corneal incision is approximately 2.0 mm from the superior limbus.

20. The method according to claim 15, wherein the end point is approximately 0.5 mm past the far end of the first incision.

21. The method according to claim 15, wherein the creating the second corneal incision comprises using scissors to open the roof of the tunnel.

22. The method according to claim 15, wherein the creating the second corneal incision comprises:
inserting a blade into the tunnel such that the blade is substantially parallel to the roof of the tunnel;
rotating the blade approximately 90 degrees such that a cutting edge of the blade faces the roof of the tunnel; and
using the cutting edge of the blade to open the roof of the tunnel.

23. The method according to claim 15, wherein during the creating of the first incision, a blade of an instrument used to create the first incision is angled toward an anterior presentation with respect to the eye.

24. A corneal paracentesis T-incision, comprising:
a clear corneal incision creating a tunnel from a surface of the clear cornea to the anterior chamber; and
a second incision, creating flaps and opening a roof of the clear corneal incision tunnel to an end point past a far end of the clear corneal incision tunnel.

25. A corneal implant implanted in a paracentesis T-incision, the paracentesis T-incision including a clear corneal incision creating a tunnel from a surface of the clear cornea to the anterior chamber, and a second incision, creating flaps and opening a roof of the clear corneal incision tunnel to an end point past a far end of the clear corneal incision tunnel, wherein the implant has a body connecting a cap and a foot, the foot is disposed within the anterior chamber, a portion of the foot is disposed beneath a floor of the clear corneal incision tunnel, and the flaps are tucked under the cap.
